(19) United States
(12) Patent Application Publication
(10) Pub. No.: US 2006/0142686 A1
(43) Pub. Date: Jun. 29, 2006

(54) Tissue injury protection medical bridge bandages

(76) Inventor: William Slade Morse, Franklin, NC (US)

Correspondence Address: William S. Morse, P.O. Box 112, Franklin, NC, 28744, US

(21) Appl. No.: 11/025,894

(22) Filed: Dec. 28, 2004

(51) Int. Cl. A61F 15/00; A61F 13/00

(52) U.S. Cl. 602/42; 602/41; 602/43; 602/44; 602/52

(57) ABSTRACT

The Tissue Injury Protection Bridge Bandages are better protection for wounds, infections, surgery, burns and other damaged body tissues. The tape-on cushioned and flexible micro ventilated sterile polymer bandage allows filtered air flow to the wound facilitating the natural body healing process. The new method and material will eliminate smothering wounds with gauze and secondary damage to the tissue when changing bandages. The TIP Bridge Bandages will be of considerable advantage over bandages used today in emergency services, clinics, operating and recovery procedures.

### BACKGROUND OF THE INVENTION

All of us have experienced or observed injured, post operation and sick people being bandaged by various medical professionals. It occurred to me that there is a better way to protect the injured body—hence the prefix TIP, acronym for tissue injury protection medical bridge bandage. With TIP Bridge Bandages one gives better protection and avoids tearing tender tissue when changing bandages thus allowing the wound to heal sooner.

### BRIEF SUMMARY OF THE INVENTION

Today we are taught by medical practitioners to place a gauze pad on an open wound and wrap it with gauze. This smothers the healing area and the healing area is torn loose on each subsequent changing of the bandage. This method delays healing and causes unnecessary discomfort for the patient.

Tissue Injury Protection Medical Bridge Bandages are designed to eliminate these existing problems by providing better bandages that will provide better protection to the area and allow healing to occur earlier with less discomfort to the patient. The sterile porous polymer material used in the manufacture of the TIP Bandages permit filtered air flow (see section for Drawings) to the wounds thereby facilitating the healing process.

### BRIEF DESCRIPTION OF THE SEVERAL VIEWS OF THE DRAWING

Page 1—Typical Views

FIG. 1 Bridge Bandage as taken from box sent by manufacturer: sealed in sanitary clear plastic film.

FIG. 2 Standard 2″×6″, 4″×6″, 6″×8″ and a variety of other sizes depending on market needs and special orders.

FIG. 3 This view shows how the tape-on strips fit on to the bandage with peel off tape guard paper.

Page 2—Exploded View: Typical Construction

FIG. 4 Tape Guard.

FIG. 4 Tape on one side only.

FIG. 4 Top cover, poly-breathe material.

FIG. 4 Vinyl bridge screen #0.5 MM gauge.

FIG. 4 Bottom cover, poly-breathe material.

FIG. 4 Tube cover, poly-breathe material.

FIG. 4 Vinyl tube screen #0.25 MM gauge.

FIG. 4 Open to allow breathing.

FIG. 4 Poly-breathe material fill around tube screen.

Note: Tape strips are anchored to top fabric in an alternating pattern with three on either side (see FIG. 8). The tubes are anchored to the bridge as shown in FIG. 2. All seams and the four tube ends are sealed by automated heat-pressure seal units.

Page 3—Application of the TIP Bridge Bandages

FIG. 5 Initial dressing in the field such as auto accidents and other trauma events often result in open wounds which require packing to slow or stop bleeding until the patient reaches a treatment center where surgeons can make repairs to stop the bleeding. The tape-on Bridge bandages will be very handy in such situations.

FIG. 6 Post operative bandaging will be made much easier with ready made tape-on TIP Bridge Bandages which not only protect the sutured area but the tender surrounding area.

FIG. 7 Special made TIP Bridge Bandages will be available for unusual tissue damage such as burns, chemical irritation, blisters, cosmetic surgery, eye surgery and to prevent rubbing between limbs.

FIG. 8 Dressing method for improved protection and healing.

Note: The new TIP Bridge Medical Bandage improves protection by the flexible padded construction and filtered ventilation which facilitates the natural body healing process.

Page 4—Application of the TIP Bridge Bandages

FIG. 9 Abdominal

FIG. 10 Arm

FIG. 11 Leg

FIG. 12 Surgical Drain—See through view.

Page 5—Available supply at treatment centers.

FIG. 13 Emergency Room

Page 6—Gazette Picture

FIG. 14

### DETAILED DESCRIPTION OF THE INVENTION

The Tissue Injury Protection “TIP” Bridge Bandages are designed to change the present methods of dressing trauma and surgery damaged tissue in the field of medicine:

Today an injured person receives some antiseptic or antibacterial medicine if the wound is open, upon which is placed a gauze pad followed by gauze wrapping which “smothers” the mending tissue. Within an hour or so the patient is further examined at an emergency room for possible surgery. The dressing is repeated and again for several days. Each time the dressing is changed the soiled gauze is pulled off of the healing tissue causing further damage to the tissue. This delays the healing of the wound and causes extended discomfort for the patient.

The natural protective film the body generates over the open wound is part of the body's way of protecting itself from bacteria and other contaminates. When this film is punctured or invaded the wound is vulnerable to infections. Coagulation of the blood, also, is a natural body function in the healing process. When changing bandages as medical workers are trained to do today the protective film is usually torn off the wound by removing the gauze causing the fluids to run and bleeding and pain to the patient.

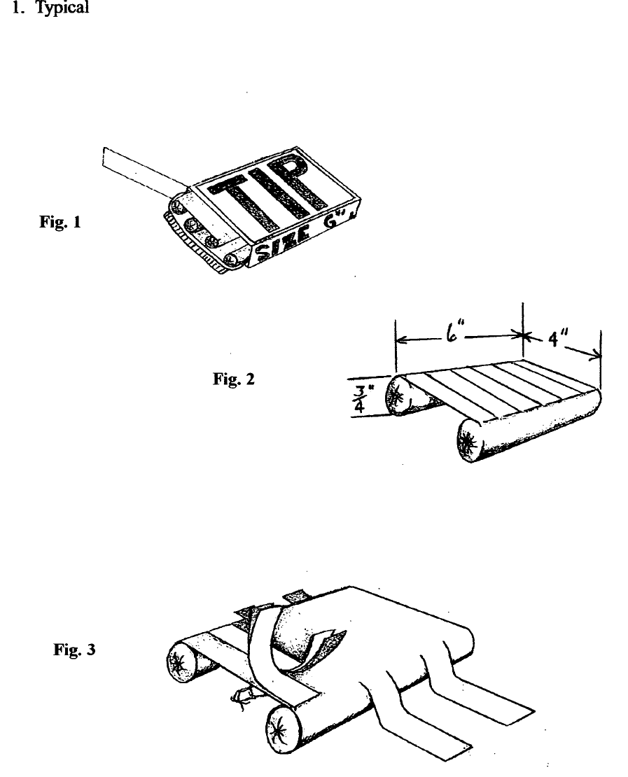

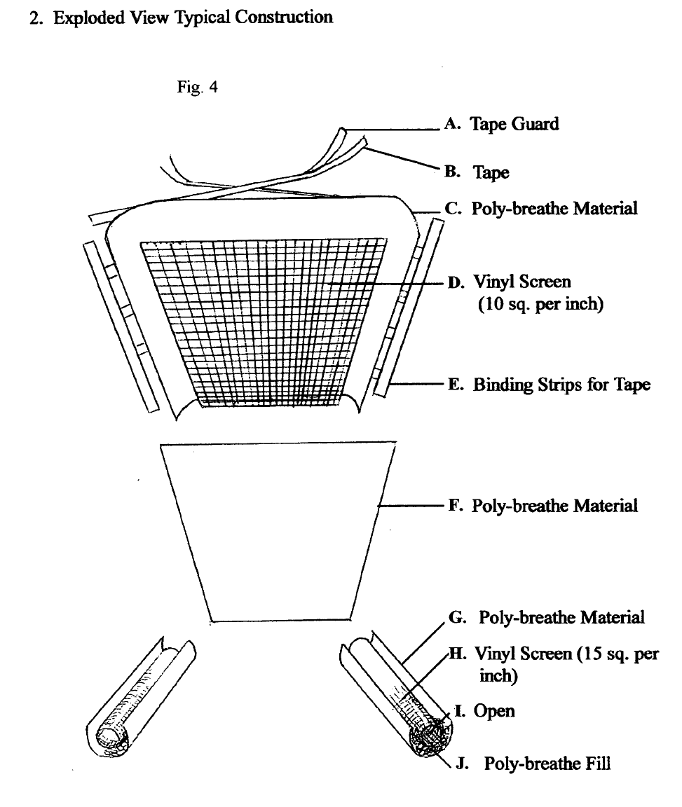

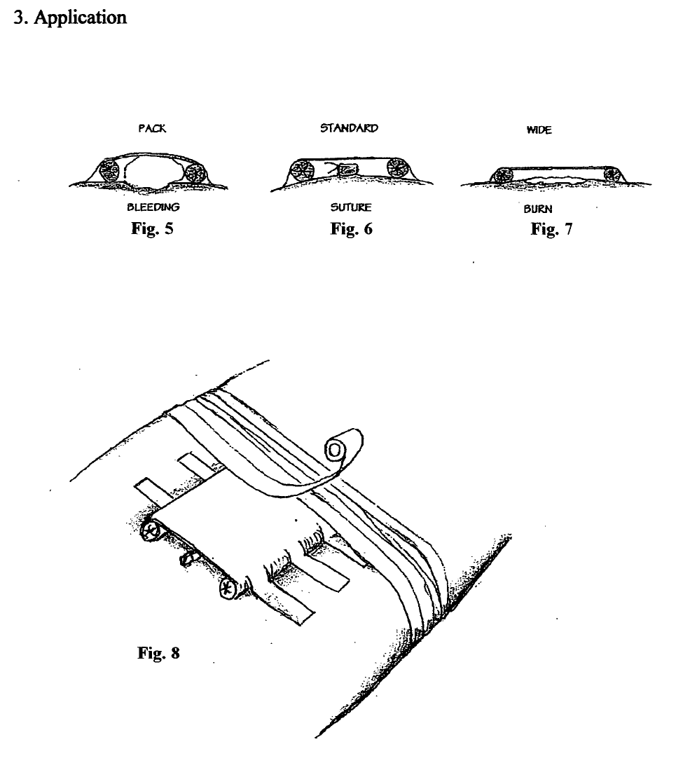

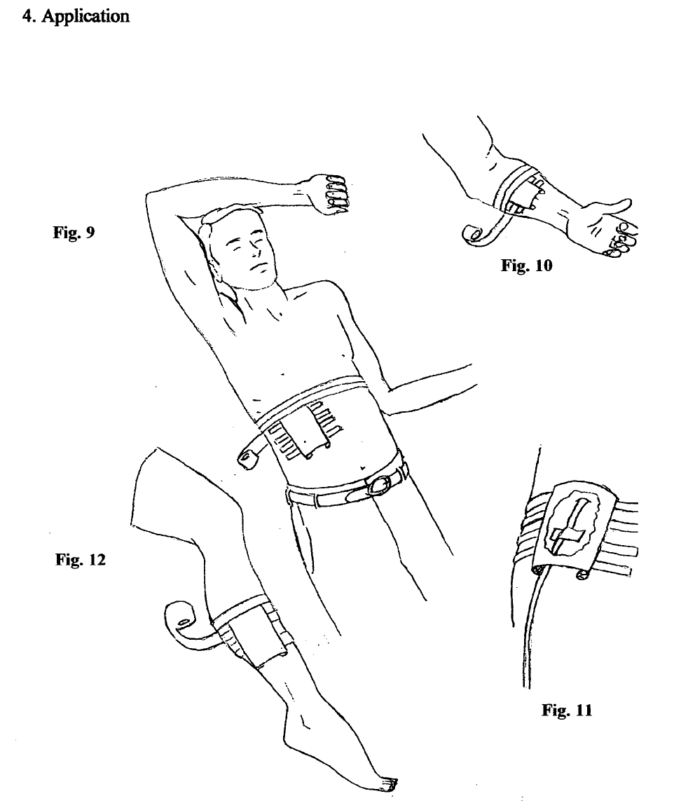

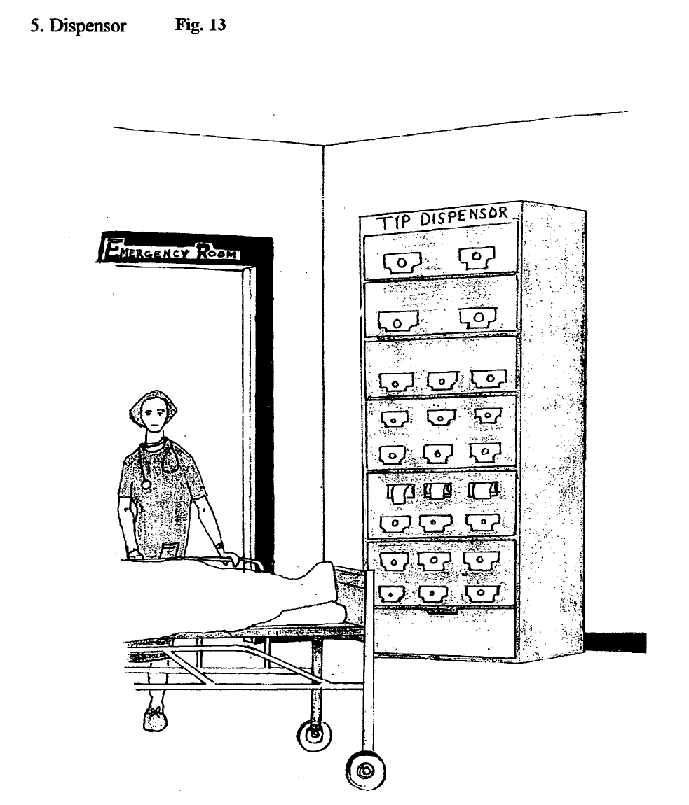

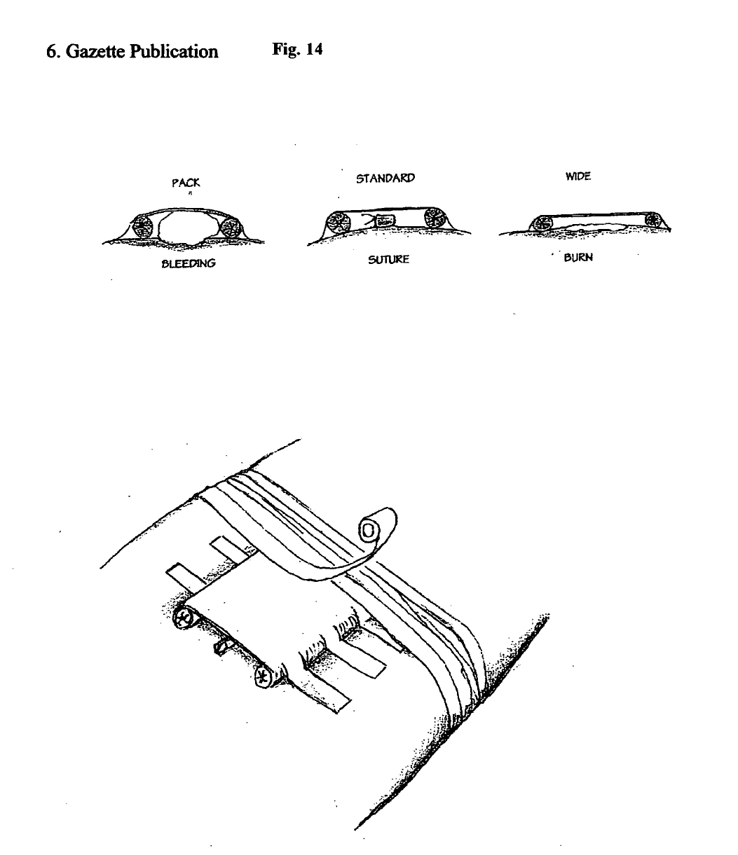

With the new TIP Bridge Bandages changes can be made without damage to the wound, eliminating the secondary danger of infection and with much less discomfort to the patient. Almost no retraining will be required for experienced technicians to understand why and how of the advantages and simplicity of using the new TIP “tape-on” Bridge Bandages (see FIGS. 5, 6, 7. 8, and FIGS. 9, 10, 11, 12). It is clear to see that gauze pads and wrappings are not placed directly on the damaged tissue unless to stop bleeding before reaching a medical treatment center where the open wound can be closed surgically. Further the gauze wrappings are limited to helping hold the bandage in place for active people. Very little gauze wrapping is needed for patients who are not ambulatory. Limited gauze wrappings allow the tissue to “breath” (see FIG. 4 through 11), a factor contributing to the body's natural healing process. The TIP Bridge Bandages will be made of porous sterile material (see FIG. 2) to provide filtered air flow to the damaged tissue. The tape-on advantage of the Bridge Bandages (see FIGS. 1, 2, 3) are especially adaptable in the field for a wide variety of accidents such as auto accidents, sports injuries (see FIGS. 9, 10, 11), operating room procedures (see FIG. 6) and recovery aids (see FIG. 11).

The manufacturing of the TIP Bridge Medical Bandages is well in the capability of several Fortune 500 corporations. The polymer industry has been producing a variety of related products used for other purposes. There are several recently developed computer controlled injection molding machines such as Boy, and other LSR micromolding machines. It was a three billion dollar industry in 2003. A team of engineers from any one of these companies could make a satisfactory model of several bandages in a month or two. Coordinating internal departments, subsidiary suppliers, advertising and marketing outlets would take at least six months to a year. Substantial production would take a year or more and production adjustments to meet the growing demand from a wide variety of medical markets would require continual adjustments for several years.

1. What I claim as my invention is a new type of medical bandage which can be used in a wide variety of procedures.
First, the cushioned, flexible tape-on designs of the Bridge Bandage provide better protection to the primary damaged tissue (accident, infection, surgery, etc.) and the surrounding affected area. The handy tape-on cushion bandage gives added protection from bumping and clothes rubbing in normal and vigorous activity.
Secondly, the micro-perforated sterile polymer material allows filtered air flow to the damaged tissue facilitating the natural body healing process.
Thirdly, the new method and material eliminates smothering with gauze and causing secondary damage to the tissue when changing bandages.
Please see the drawings (FIG. 1-A through 4-D) included in this application.
